(19) United States
(12) Patent Application Publication
(10) Pub. No.: US 2022/0143319 A1
(43) Pub. Date: May 12, 2022

(54) DRUG DELIVERY DEVICE WITH SMART GRIP

(71) Applicant: AMGEN INC., Thousand Oaks, CA (US)

(72) Inventor: Jimmie L. Ward, Camarillo, CA (US)

(21) Appl. No.: 17/602,148

(22) PCT Filed: Apr. 6, 2020
(86) PCT No.: PCT/US20/26807

§ 371 Date: Oct. 7, 2021

Related U.S. Application Data
(60) Provisional application No. 62/831,473, filed on Apr. 9, 2019.

(51) Int. Cl. A61M 5/31; A61M 5/315; A61M 5/20

(52) U.S. Cl. CPC A61M 5/3137; A61M 5/3158; A61M 5/20; A61M 2205/586; A61M 2005/3126; A61M 2205/583; A61M 2205/13; A61M 2005/3139; A61M 2230/63; A61M 2205/581; A61M 2205/587

(57) ABSTRACT

A drug delivery device includes an injector and an accessory grip. The injector includes an injector housing defining a housing body having a proximal end, a distal end, and a longitudinal axis extending therebetween, a needle assembly at least partially disposed within the injector housing at the proximal end, and a drive assembly at least partially disposed within the housing. The needle assembly includes a syringe barrel containing a medicament and a needle or a cannula. The drive assembly urges the medicament through the needle or cannula. The accessory grip defines a grip shell having proximal and distal ends, and a body extending therebetween. The proximal end includes a first opening dimensioned to receive a first portion of the injector housing, and the distal end of the grip shell includes a second opening that receives a second opening dimensioned to receive a second portion of the injector housing.

### CROSS-REFERENCE TO RELATED APPLICATION

Priority is claimed to U.S. Provisional Patent Application No. 62/831,473, filed Apr. 9, 2019, the entire contents of which are hereby expressly incorporated by reference.

### FIELD OF DISCLOSURE

The present disclosure generally relates to drug delivery devices and methods. More particularly, the present disclosure relates to improved drug delivery devices having advanced compatibility features.

### BACKGROUND

Drugs are administered to treat a variety of conditions and diseases. Autoinjectors and on-body injectors (e.g., pen style autoinjectors) offer several benefits in delivery of medicaments and/or therapeutics. One of the benefits can include simplicity of use, as compared with traditional methods of delivery using, for example, conventional syringes. Autoinjectors may be used to deliver a number of different drugs having varying viscosities and/or desired volumes.

A length of tolerable injection times for patients using handheld autoinjectors is often limited by the patient's ability to sustainably and comfortably grip and control the device while maintaining a stable placement and orientation of the device on the patient's injection site. Some patients may have a tendency to remove the device prior to completion of injection in an effort to determine whether the injection was in fact completed. Further, some patients may have reduced manual dexterity and/or cognitive ability, which may make self-injection of drugs physically demanding and can result in treatment noncompliance. Additionally, family members may often serve as caregivers, and they may not be familiar with the autoinjector product and may themselves suffer from a loss of sensation, dexterity and/or any other flexing or grasping issues in their hands or bodies when attempting to assist with drug administration.

Existing autoinjector designs may be unstable and require a user to hold the device steadily and carefully in place throughout the injection process in order to effectively and properly administer the drug. Oftentimes, premature removal of the device from the delivery site can result in an incomplete dosage being delivered due to the drug spraying onto the skin surface. Further, existing single-use, pen style autoinjectors can provide mechanical feedback mechanisms, but due to their limited space, lack electronics and/or data management capabilities. Autoinjectors that do include these capabilities may be highly complex and expensive to design, manufacture, package, store, ship, and dispose of due to being used in single-use applications.

As described in more detail below, the present disclosure sets forth smart grip systems for delivery devices embodying advantageous alternatives to existing systems and methods, and that may address one or more of the challenges or needs mentioned herein, as well as provide other benefits and advantages.

### SUMMARY

In accordance with a first aspect, a drug delivery device includes an injector and an accessory grip. The injector includes an injector housing defining a housing body having a proximal end, a distal end, and a longitudinal axis extending therebetween, a needle assembly at least partially disposed within the injector housing at the proximal end, and a drive assembly at least partially disposed within the housing. The needle assembly includes a syringe barrel containing a medicament and a needle or a cannula. The drive assembly urges the medicament through the needle or cannula. The accessory grip defines a grip shell having proximal and distal ends, and a body extending therebetween. The proximal end includes a first opening dimensioned to receive a first portion of the injector housing, and the distal end of the grip shell includes a second opening that receives a second opening dimensioned to receive a second portion of the injector housing.

In some examples, the device further includes an injector housing latching member disposed on the housing body and a grip latching member disposed on the grip shell. The grip latching member secures to the injector housing latching member to secure the injector housing to the accessory grip. In some approaches, the injector housing latching member is in the form of at least one depression formed on the shell. Further, the grip latching member may be in the form of at least one finger member configured to be inserted into the at least one depression. The at least one depression may form a dosage window on the injector housing. In these examples, the accessory grip may additionally include at least one viewing window that is aligned with the dosage window.

In some approaches, the grip shell may further define a throughbore that extends between the first and second openings. The throughbore is dimensioned to receive the injector in a manner that the proximal end of the injector housing is exposed through the first opening of the grip shell and the distal end of the injector housing is exposed through the second opening of the grip shell.

In some examples, the accessory grip may further include at least one electronic device at least partially disposed within the grip shell. For example, the accessory device may be in the form of a display, a lighting system, a skin sensor, a communications module, a motion sensor, or an electromechanical feedback mechanism. The electronic device may be in communication with the injector.

In some forms, the accessory grip is in the form of a tubular clamshell that extends in a direction along the longitudinal axis of the injector housing. In other examples, the accessory grip may be in the form of an elongated dome-shaped clamshell that has a curved upper gripping surface. In some examples, the proximal end of the grip portion may further include a planar contact surface.

In accordance with a second aspect, an accessory grip for a drug delivery device includes a grip shell having a proximal end, a distal end, and a body extending between the proximal end and the distal end thereof, a first opening formed at the proximal end of the grip shell, a second opening formed at the distal end of the grip shell, and a grip latching member disposed on the grip shell. The first opening is dimensioned to receive a first portion of a drug delivery device, and the second opening is dimensioned to receive a second portion of the drug delivery device. The first opening and the second opening are in communication with each other via a throughbore extending therebetween. The grip latching member is adapted to couple to the drug delivery device to secure the accessory grip to the drug delivery device.

### BRIEF DESCRIPTION OF THE DRAWINGS

The above needs are at least partially met through provision of the drug delivery device having a smart grip described in the following detailed description, particularly when studied in conjunction with the drawings, wherein:

FIG. 1 illustrates a perspective view of an example injector in accordance with various embodiments;

FIGS. 2a and 2b illustrate front elevation and perspective views, respectively, of an example injector having a smart grip coupled thereto in accordance with various embodiments;

FIG. 3 illustrates a top plan view of the example injector having a smart grip coupled thereto of FIGS. 2a and 2b in accordance with various embodiments;

FIG. 4 illustrates a cross-sectional view of the example injector having a smart grip coupled thereto of FIGS. 2a-3 in accordance with various embodiments;

FIG. 5 illustrates a schematic view of the example injector having a smart grip coupled thereto of FIGS. 2a-4 in accordance with various embodiments;

FIG. 6 illustrates the example injector having a smart grip coupled thereto of FIGS. 2a-5 being administered at a first example location in accordance with various embodiments;

FIG. 7 illustrates the example injector having a smart grip coupled thereto of FIGS. 2a-5 being administered at a second example location in accordance with various embodiments;

FIG. 8 illustrates a perspective view of a second example smart grip in accordance with various embodiments;

FIG. 9 illustrates a perspective view of a third example smart grip in accordance with various embodiments;

FIG. 10 illustrates a perspective cross-sectional view of an interior of the example smart grip of FIG. 9 depicting an example guide region in accordance with various embodiments;

FIGS. 11a-11c illustrate front elevation views of the example smart grip of FIGS. 9 and 10 depicting an example locking mechanism in accordance with various embodiments;

FIG. 12 illustrates a front elevation view of the example smart grip of FIGS. 9-11c in a post-injection configuration in accordance with various embodiments;

FIGS. 13a and 13b illustrate perspective views of a fourth example smart grip in accordance with various embodiments;

FIG. 14 illustrates a front elevation partial cross-sectional view of the example smart grip of FIGS. 13a and 13b in accordance with various embodiments;

FIG. 15 illustrates a rear perspective view of the example smart grip of FIGS. 13a-14 in accordance with various embodiments;

FIG. 16 illustrates a perspective cross-sectional view of an interior of the example smart grip of FIGS. 13a-15 depicting an example guide region in accordance with various embodiments;

FIG. 17 illustrates a perspective view of an example locking mechanism for use with the example smart grip of FIGS. 13a-16 in accordance with various embodiments;

FIGS. 18a-18c illustrate perspective views of the example smart grip of FIGS. 13a-17 coupling to an example drug delivery device in accordance with various embodiments;

FIG. 19 illustrates a front elevation view of an example release assembly of the example smart grip of FIGS. 13a-18 in accordance with various embodiments;

FIG. 20 illustrates a perspective view of an example release mechanism of the example release assembly of FIG. 19 in accordance with various embodiments;

FIG. 21 illustrates a side elevation partial cross-sectional view of the example smart grip of FIGS. 13a-20 in accordance with various embodiments;

FIGS. 22a and 22b illustrate perspective views of a release procedure for the release assembly of the example smart grip of FIGS. 13a-21 in accordance with various embodiments; and

FIGS. 23a and 23b illustrate perspective views of the example release assembly of FIGS. 20-22b in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

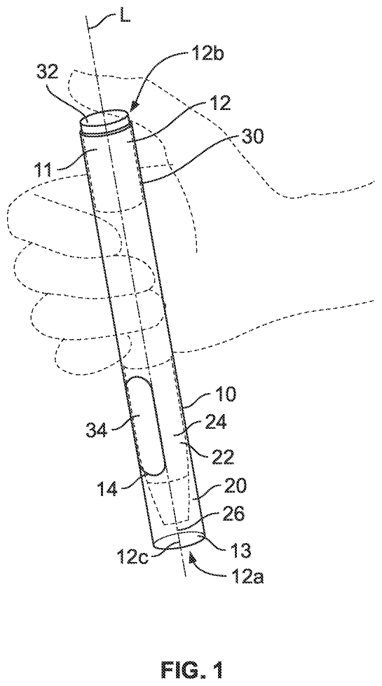

Generally speaking, pursuant to these various embodiments, a drug delivery device (e.g., an autoinjector or other injector) is coupled to a shell that at least partially surrounds the injector to increase a graspable surface area. This shell can accommodate any number of additional smart features including electronics and connectivity to further enhance the drug administration experience. As illustrated in FIG. 1, an example injector 10 generally includes an injector housing 11 defining a body 12 that includes a proximal end 12a, a distal end 12b, and a longitudinal axis “L” extending between the proximal and distal ends 12a, 12b. The body 12 further includes a generally planar contact surface 13 positioned at the proximal end 12a thereof. Further, the body 12 includes an injector housing latching member 14 which, in the illustrated example, includes a recessed surface adjacent to a dosage window 34 of the injector. In other forms, however, the latching member 14 can take the form of at least one depression, ledge, indentation, barb or other suitable structure formed on or carried by the body 12. In other forms, the injector housing latching member may be in the form of any number of projections disposed on the shell 12.

A needle assembly 20 is at least partially disposed within the body 12 at or near the proximal end 12a thereof, and includes a syringe barrel 22 that contains a medicament 24 and a needle or a cannula 26 that is used to inject the medicament 24 to a patient. The body 12 defines an opening 12c at the proximal end 12a that is dimensioned to accommodate the needle or the cannula 26. In some examples, the needle or cannula 26 may be initially positioned within the body 12 prior to activation, and may protrude through the opening 12c during drug delivery.

A drive assembly 30 is also at least partially disposed within the body 12 and is operably coupled to the needle assembly 20. The drive assembly 30 may include an actuator button 32 positioned at or near the distal end 12b of the body 12 that initiates actuation of the drive assembly 30. Generally speaking, in use, a user places the contact surface 13 of the body 12 against their skin (e.g., on their leg or their stomach) and actuates the actuator button 32. This actuation causes a drive mechanism (in the form of a spring, a motor, a hydraulic or pressurized mechanism, etc.) of the drive assembly 20 to exert a driving force on the needle assembly 20 that causes the needle or cannula 26 to be inserted through the opening 12c of the body 12 and into a patient, and that further causes the medicament 24 to be urged from the syringe barrel 22, out the needle or cannula 26, and to the patient. In some versions, the patient may manually insert the needle or cannula 26, and actuation of the drive mechanism 30 only includes causing the medicament 24 to be urged from the syringe barrel 22, out the needle or cannula 26, and to the patient.

The injector 10 may include any number of additional features and components that may assist and/or enhance the functionality of the device, such as, for example, any number of dosage windows 34 positioned at or near the syringe barrel 22 to provide a visual indication of the remaining quantity of drug during administration. As mentioned above, in some examples, the injector housing latching member 14 may define a portion or indented surface of the dosage window 34. The injector 10 may additionally include one or more electronic modules that are coupled to the body 12, the needle assembly 14, the drive assembly 20, and/or any other components of the injector 10. Further, the injector 10 may also include any number of safety mechanisms such as needle shields, retraction mechanisms, damping mechanisms, and the like. Other examples of desired mechanisms, subassemblies, and/or components are possible.

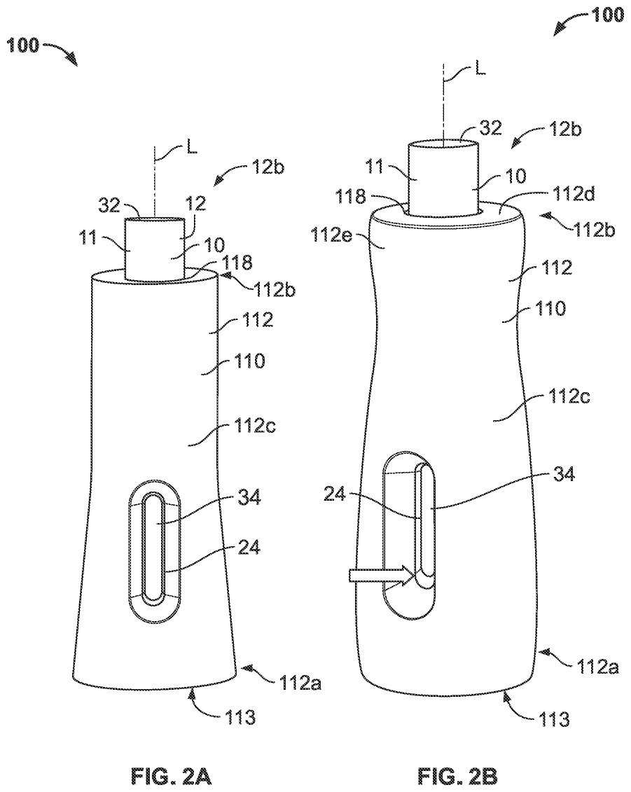

Turning to FIGS. 2a-5, a drug delivery system 100 includes an injector (e.g., the aforementioned injector 10) and an accessory grip 110 operably coupled to the injector 10. The accessory grip 110 defines a grip shell 112 having a proximal end 112a, a distal end 112b, and a body 112c extending between the proximal and distal ends 112a, 112b. As seen in FIG. 4, an outer radial dimension of the body 112c is larger than an outer radial dimension of the actuator body 12, thus increasing the size of the grasping surface for a user to securely hold the system 100. That is, the increased radial dimension of the body 112c provides for a thicker device, which may more easily be grasped by patients with lower dexterity and/or reduced muscle strength. The proximal end 112a of the grip shell 112 includes a first opening 116, and the distal end 112b of the grip shell 112 includes a second opening 118. The first and second openings 116, 118 communicate with each other by way of a throughbore 119 that extends through the grip shell 112 for receiving the injector 10 in a coaxial manner. That is, a longitudinal axis of the throughbore 19 is coaxially aligned with the longitudinal axis L of the injector 10 when the injector 10 is installed in the grip shell 112. While the disclosed version of the accessory grip 110 has an inner wall defining the throughbore 119, in some examples, the accessory grip 110 may not include this inner wall. In these examples, the throughbore 119 still connects the first and second openings 116, 118.

Further, the accessory grip 110 includes grip contact surface 113 disposed at the proximal end 112a thereof as well as a grip latching member 114 disposed on and/or coupled to the grip shell 112 (e.g., near the proximal end 112a thereof). The grip contact surface 113 is a substantially planar end surface of the grip shell 112 at the proximal end 112a, and includes a radial dimension defined by the radial dimension of the shell 112. As such, the grip contact surface 113 includes a surface area that extends radially beyond the contact surface 13 of the injector 10. As such, the grip contact surface 113 and the contact surface 13 collectively define a surface area that is much larger than just the contact surface 13 of the injector to facilitate skin contact and engagement, which helps prevent tipping and misalignment during use against a patient's skin.

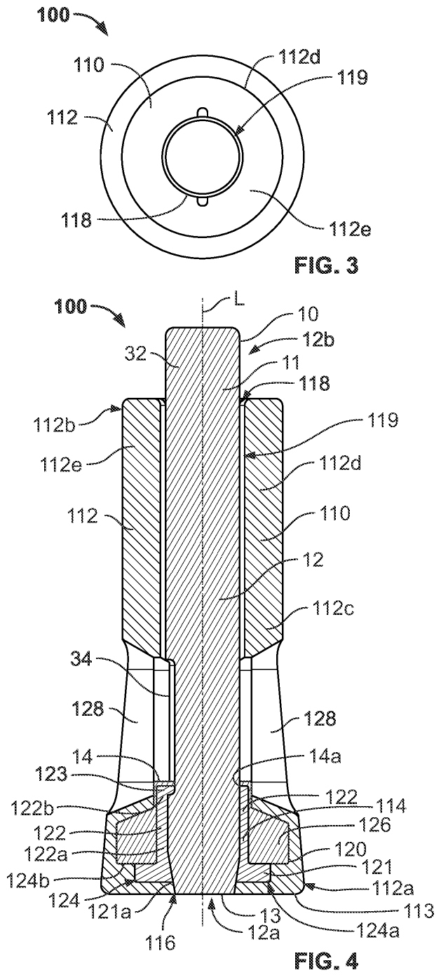

As illustrated in FIG. 3, the grip shell 112 may be in the form of a clamshell that includes a first portion 112d and a second portion 112e. The first and the second portions 112d, 112e of the clamshell may wrap around and couple together on opposite sides of the injector 10 via any suitable coupling mechanism or mechanisms such as, for example, a friction-fit connection, a releasable hinged connection, a latch connection, a magnetic connection, adhesive, a hook and loop fastener, and the like. Other examples are possible. The grip shell 112 may have a contoured, varying, and/or tapered ergonomic outer profile to provide a comfortable grip for all users including users having various hand sizes. The grip shell 112 may additionally include rubberized and/or elastomeric grip surfaces to assist in use.

The first opening 116 of the grip shell 112 is dimensioned to accommodate a first portion (e.g., the proximal end 12a) of the injector 10. For example, the first opening 116 of the grip shell 112 may have a dimension (e.g., a diameter and/or a width) between approximately 0.1 mm and approximately 5 mm to accommodate the needle or cannula 24 of the injector during drug delivery. In other examples, the first opening 116 of the grip shell may be of a larger dimension between approximately 5 mm and approximately 50 mm to accommodate all or a portion of a width and/or a diameter of the body 12. Other examples are possible. In the illustrated example, and as shown in FIG. 4, the contact surface 13 of the body 12 is coplanar with the grip contact surface 113 to collectively provide increased skin contact area as described above. However, in other examples, the contact surface 13 of the body 12 may be recessed inwardly or protrude outwardly relative to the grip contact surface 113.

The second opening 118 of the grip shell 112 is dimensioned to accommodate a second portion (e.g., the distal end 12b) of the injector 10. For example, the second opening 118 of the grip shell 112 may have a dimension (e.g., a diameter and/or a width) between approximately 5 mm and approximately 50 mm to accommodate all or a portion of the distal end 12b of the body 12. Other examples are possible. In the illustrated example, the grip shell 112 includes a longitudinal dimension that is smaller than a longitudinal dimension of the injector 10 such that at least the actuator button 32 protrudes outwardly through the second opening 118, thereby allowing a user to actuate the drive assembly 30 to deliver the medicament 24 via the first opening 116.

The grip latching member 114 operably couples to the injector housing latching member 14 to secure the injector 10 to and/or within the accessory grip 110. More specifically, as illustrated in FIG. 4, the grip latching member 114 is in the form of a disk 120 that defines a base 121 having an opening 121a, and further having any number of axially-extending resilient fingers 122 protruding upwardly (in the illustrated orientation) therefrom. The finger or fingers 122 include a proximal end 122a positioned adjacent to the disk 120 and a distal end 122b away from the disk 120 that defines a tab 123. In some examples, the finger or fingers 122 may be in the form of a single concentric ring that extends from the disk 120. The disk 120 may be disposed within and/or formed integrally with the grip shell 112. Further, in some examples, the finger or fingers 122 may have a resting position or configuration where the tab 123 is disposed radially inwardly relative to the opening 116. Put differently, the tab 123 may extend into the area defined by the opening 116. In these examples, and as will be discussed in further detail below, the finger or fingers 122 may then be flexed or be urged outwardly upon placement of the injector 10 into the clamshell 112d, 112e.

With continued reference to FIG. 4, each portion of the clamshell 112d, 112e defines an annular or partially annular disk cavity 124 that forms a first ledge 124a and a second ledge 124b. The first ledge 124a of the disk cavity 124 is dimensioned to receive the disk 120, and may form a friction-fit coupling therebetween. The second ledge 124b of the disk cavity 124 is dimensioned to receive a guide or positioning ring 126, which, in some examples, may be constructed from a resilient material. In some examples, the guide ring 126 may exert a radially inward force on the finger or fingers 122 when the first and second portions of the clamshell 112d, 112e are coupled together.

Generally, the injector 10 may be inserted through the opening 121a of the base 121. In some examples, the disk 120 and the guide ring 126 may each include discrete portions that couple to each-other via any number of approaches and/or mechanisms. In these examples, the discrete portions of the disk 120 and the guide ring 126 may first be inserted into the disk cavity 124 formed in each of the clamshell portions 112d, 112e, and subsequently, the injector 10 may be positioned within the grip shell 112. Upon closing the first and second portions 112d, 112e of the grip shell 112, the guide ring 126 and/or the finger or fingers 122 may exert a securing force inwardly towards the injector 10. More specifically, the resilient finger or fingers 122 may exert an inward clamping force to cause the tab 123 positioned at the distal end 122b thereof to engage the injector housing latching member 14 (e.g., the depression at least partially defining the dosage window 34) to secure the grip 110 to the injector 10, thus limiting and/or restricting relative movement therebetween. In these configurations, the tab 123, which is positioned axially above the injector housing latching member 14, prevents the injector 10 from moving in an upward axial direction relative to the accessory grip 110. Further, in some examples, the resilient finger or fingers 122 themselves may exert an inward clamping force on the body 12 of the injector 10 such that the finger or fingers 122 frictionally engage the body 12. In some examples, the injector housing latching member 14 may further include a radial groove 14a or detent that receives the tab 123 of the finger or fingers 122 to limit and/or restrict relative axial, longitudinal, and/or rotational movement between the injector 10 and the grip shell 112. In these examples, the tab 123 may nest within the radial groove 14a or detent and may remain nestled therein via the inward urging of the resilient finger or fingers 122. The finger or fingers 122 may additionally include a release mechanism that opens the grip shell 112. In some examples, the engagement between the tab 123 and the housing latching member 14 may be a frictional engagement, but in other examples, an active locking mechanism (not illustrated) may be used to prevent relative movement of the injector and the grip shell 112.

In some approaches, the injector 10 may first be inserted through the opening 121a of the base 121, and the guide ring 126 may be positioned on the base 121. The outer diameter of the proximal end 12a of the body 12 may be dimensioned to outwardly urge or “splay” the finger or fingers 122, thereby creating a friction-fit connection between the disk 120 and the injector 10. The first and second portions of the clamshell 112d, 112e may then be closed around the injector 10, the disk 120, and the guide ring 126 while the disk 120 and the guide ring 126 are aligned with the first and second ledges 124a, 124b of the disk cavity 124 to secure the grip 110 to the injector 10 via the finger or fingers 122.

So configured, when the injector 10 is at least partially disposed within the grip 110, the grip contact surface 113 is increased relative to the contact surface 13 of the body 12. This increased contact surface results in increased stability during drug administration, and reduces the likelihood of the injector 10 inadvertently slipping or moving, which could result in the needle or cannula 26 becoming removed from the patient. Further, the increased circumference of the grip shell 112 can allow users with limited dexterity the ability to comfortably and securely hold the system 100 in a stable position while being used with a single hand. The system 100 may be provided with various grip contact surface 113 diameters to accommodate use by different classes of patients.

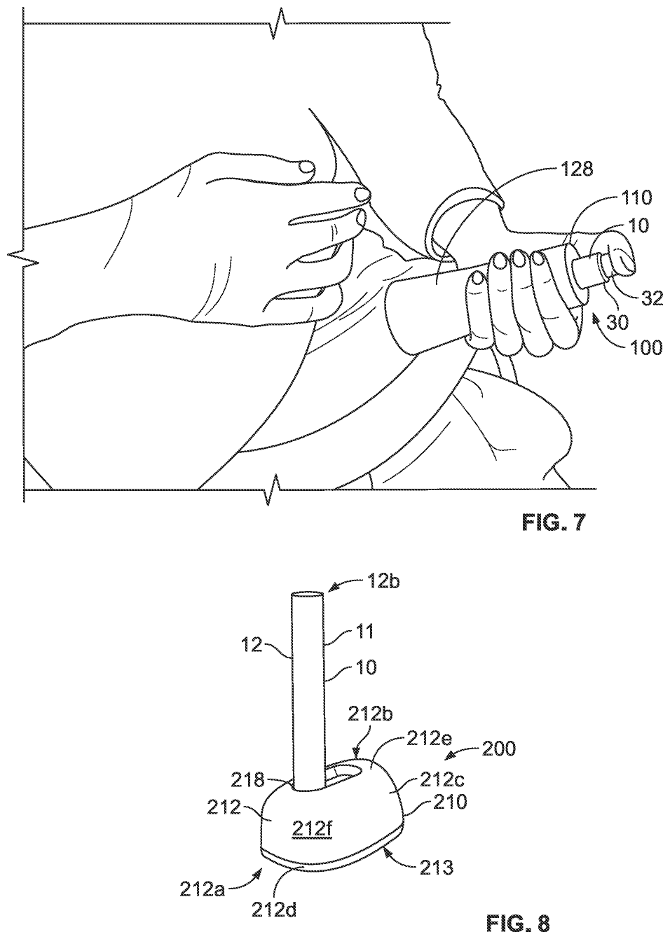

As illustrated in FIGS. 2a, 2b, 4, 6, and 7, the accessory grip 110 may include any number of viewing windows 128 that is aligned with the dosage window or windows 34 to allow a user to view the level of remaining medicament 24 in the injector 10 during administration. As illustrated in FIGS. 6 and 7, the system 100 may be grasped with one hand and oriented such that the grip contact surface 113 is positioned against a users' leg, stomach or other body part while the viewing window 128 is in view.

The system 100, and specifically the accessory grip 110, may include any number of additional features to enhance administration of the medicament 24. Because of the increased volume of the accessory grip 110 as compared to the injector 10, additional “smart” components and mechanisms may be used. The injector 10 may include a connectivity module (not shown) that allows for communication between the injector 10 and the grip 110 so that additional information may be conveyed to a user. This connectivity module may be in the form of a wired or wireless data transfer system (e.g., Bluetooth, near-field communication (“NFC”), LoRa, and the like) that transmits data to a receiver 130 disposed on or in the grip shell 112. This receiver 130 may in turn be in communication with an electronic controller (not shown) that controls operation of the additional components.

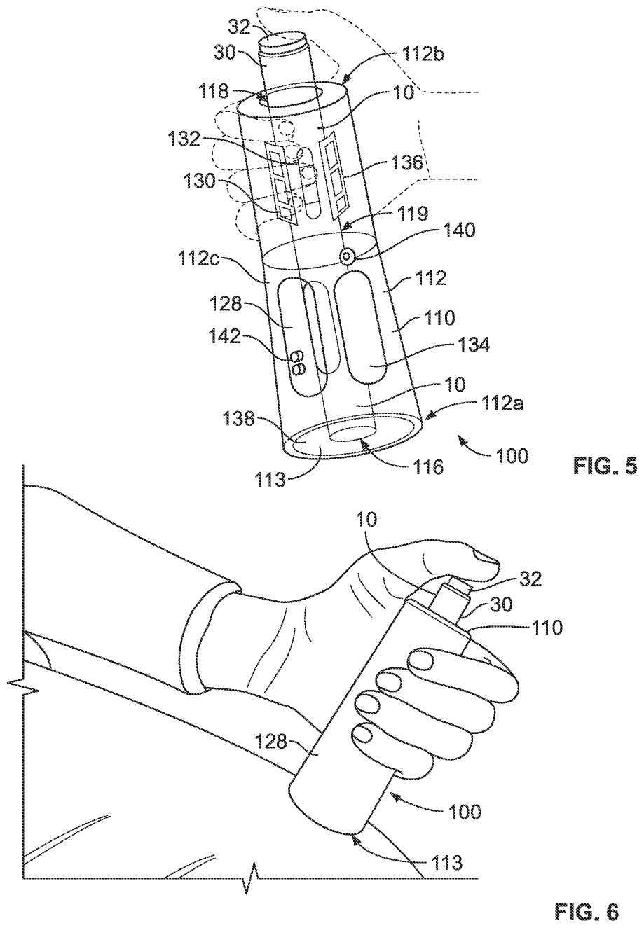

For example, as illustrated in FIG. 5, the grip shell 112 may include any number electronic devices such as a lighting system (e.g., LEDs) 132 that can provide a visual indication of the status of the injector 10. For example, the lighting system 132 may illuminate when the system 100 is ready for use, when the system 100 is properly oriented, when the sterile barrier is removed, when body contact is initiated, when the cannula or needle 26 is inserted, the position of the needle assembly 20, when the system 100 has completed administration of the medicament 24, when the system 100 encounters an error or malfunction, and the like. The grip shell 112 may further include an informational display 134 that can convey similar information in a clear, easily readable manner.

The grip shell 112 may accommodate any number of additional sensors such as, for example, a motion sensor 136 (e.g., a MEMS motion sensor) that senses movement of the system 100. This sensor 136 may then transmit sensed data to the lighting system 132 and/or the informational display 134 to indicate whether the system 100 is being held sufficiently still for proper administration. The grip shell 112 may also accommodate a skin sensor 138 that senses adequate contact to the injection site. Such a sensor 138 may be positioned at or near the grip contact surface 113. Further, the grip shell 112 may include any number of sound feedback mechanisms 140 (e.g., an electrically powered speaker) that provide an audible indication that the dose has started, completed, encountered an error, and the like. Further still, the grip shell 112 may accommodate an optical dose check 142 positioned at or near the viewing window 128 to determine the remaining level of medicament 24 in the syringe barrel 22. Any or all of these additional systems may be in communication with the controller, the lighting system 132, and or the informational display 134. The grip shell 112 may include a power source (e.g., a battery) that provides power to any number of these components.

Additional information that may be conveyed or provided to a user or healthcare professional can include compliance monitoring, enhanced feedback, time and/or location stamps, data connectivity to cloud-based systems for family members, healthcare professionals, etc., shock, vibration, or light exposure, and the like. Further, a users prescription or therapeutic regime may be displayed, characteristics of the medicament 24 (e.g., color, viscosity, and/or turbidity), geographic positioning, security and/or anti-counterfeiting information, temperature, time, and/or spatial orientation information, dosage quantities, delivery depth, dosage steps for the user to perform, and the like.

Turning to FIG. 8, a device 200 having an alternative accessory grip 210 design is provided that includes similar features as the accessory grip 110 described in FIGS. 2a-7, and thus will not be described in substantial detail. However, in this illustrated example, the accessory grip 210 is in the form of a generally elongated dome-shaped member having a track-shaped footprint. The distal end 212b of the grip shell 212 forms a curved upper gripping surface 212f that corresponds to a natural curve of a user's palm. In essence, the curved upper gripping surface 212f is approximately perpendicular to the longitudinal axis L of the injector 10, but other relative angles are possible (e.g., a relative angle of approximately 45°). So configured, a user may grasp the device 200 with their hand while allowing the injector 10 to be positioned between desired fingers. Such a shape may lower the center of gravity of the device 200 while increasing the overall area of the grip contact surface 212, thereby providing a more stable device.

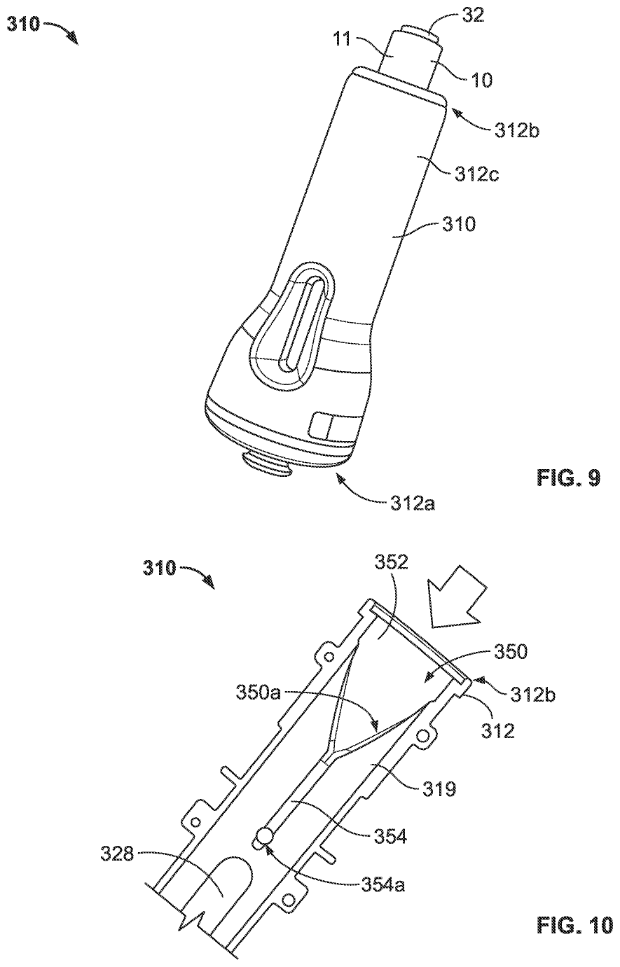

Turning to FIGS. 8-12, a device 300 having an alternative accessory grip 310 design is provided that includes similar features as the accessory grips 110, 210 described in FIGS. 2a-8, and thus will not be described in substantial detail. Like the accessory grip 110, the accessory grip 310 defines a grip shell 312 having proximal and distal ends 312a, 312b, and a body 312c extending therebetween. The accessory grip 310 further includes a viewing window 328 positioned along the body 312c of the grip shell 312 and a lighting system 332 positioned at the proximal end 312a of the grip shell 312. Advantageously, the accessory grip 310 is designed such that the viewing window 328 allows approximately 75% of the dosage window 34 of the injector 10 to be viewable by a user.

In this example, the lighting system 332 is in the form of a multi-color progress light guide that changes colors and/or light patterns to convey the status of the device. Additionally, the accessory grip 310 includes window lighting 342 which may assist with allowing a user to better see the remaining drug volume through the window 328. In some examples, the window lighting 342 may be in the form of a sensor light guide that selectively changes an illumination and/or lighting pattern during the drug administration process. The window lighting 342 may also cooperate with an optical sensor assembly to assist with viewing the drug administration progress.

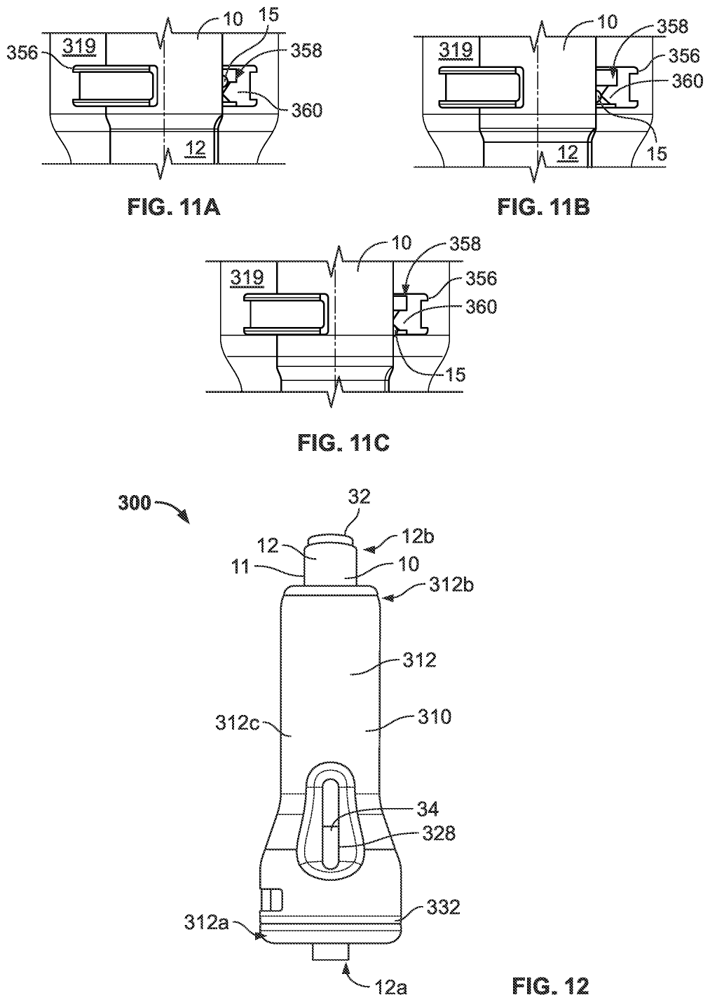

With reference to FIGS. 10-11c, the device 300 is assembled by inserting the proximal end 12a of the injector 10 downwardly into the accessory grip 310 from the distal end 312b of the grip shell 312. As illustrated in FIG. 10, the throughbore 319 of the grip shell 312 includes a guide portion 350 in the form of a funnel-shaped groove formed into the surface of the throughbore 319. The guide portion 350 includes a wide upper region 352 that tapers to a channel 354 having an end region 354a. As shown in FIGS. 11a-11c, positioned at or near the end region 354a is a locking member 356. The shell 12 of the injector housing 11 includes any number of bumps or protrusions 15 extending outwardly therefrom that engage the guide portion 350 during installation. Specifically, the throughbore 319 of the grip shell 312 is dimensioned such that the protrusion or protrusions 15 may only be inserted into the throughbore 319 when they are positioned within the guide portion 350. By providing a relatively wide upper region 352, the injector 10 can be inserted into the accessory grip 310 in a misaligned configuration because continued insertion of the injector 10 into the throughbore 319 will cause the protrusion 15 to engage a sidewall 350a of the guide portion 350 and subsequently follow the shape of the tapered upper region 352 until the protrusion 15 is inserted into the channel 354, which results in proper orientation and alignment of the injector 10.

FIGS. 11a-11c illustrate the locking engagement between the injector 10 and the accessory grip 310. As previously noted, the locking member 356 is positioned at or near the end region 354a of the channel 354. The locking member 356 may be retained by and/or secured to the accessory grip 310 by any number of suitable approaches. The locking member 356 is in the form of a flexible and/or resilient ring that includes an inner surface 358 having a protrusion 360 that extends inwardly into the opening formed by the inner surface 358. As illustrated in FIG. 11a, as the injector 10 is moved down and into the accessory grip 310, the protrusion 15 of the injector 10 contacts the protrusion 360 of the locking member 356. As shown in FIG. 11b, continued downward insertion of the injector 10 causes the protrusion 15 of the injector 10 to urge the protrusion 360 of the locking member 356 outwardly until, as shown in FIG. 11c, the protrusion 15 of the injector 10 is positioned below the protrusion 360 of the locking member 356. Once the protrusion 15 of the injector passes the protrusion 360 of the locking member 356, the resilience of the locking member 356 causes the locking member 356 to move or snap to its initial position that restricts the injector 10 from being removed from the accessory grip 310 without exerting a sufficient pulling force required to again urge the locking member outwardly 356. As illustrated in FIG. 12, upon administering the drug to the patient, the injector 10 can be readily removed by pushing the proximal end 12a of the shell 12 against a hard surface, which will cause the protrusion 15 of the injector 10 to move upwards and past the locking member 356.

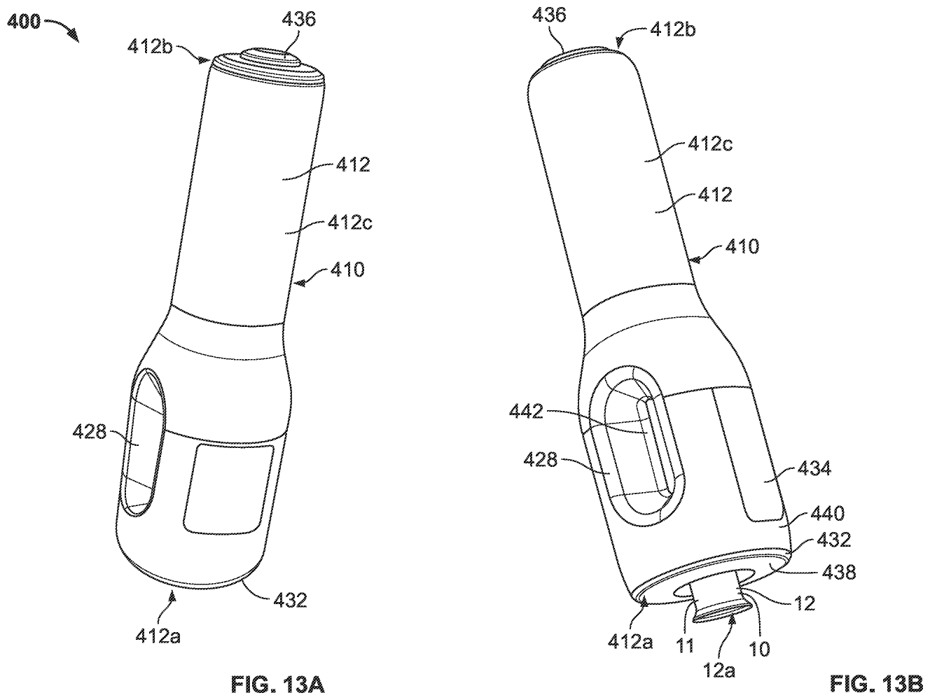

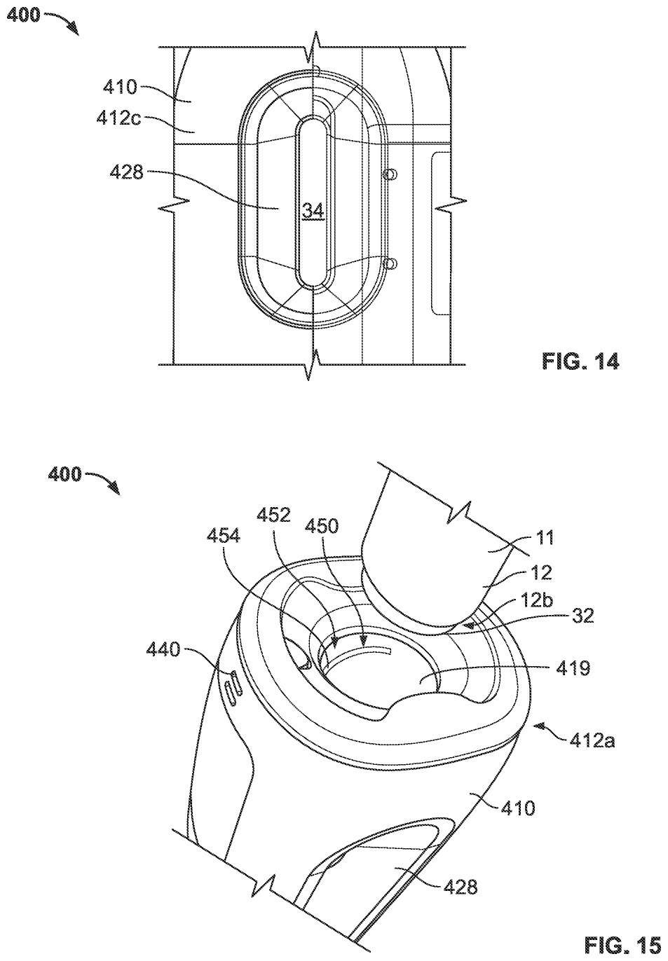

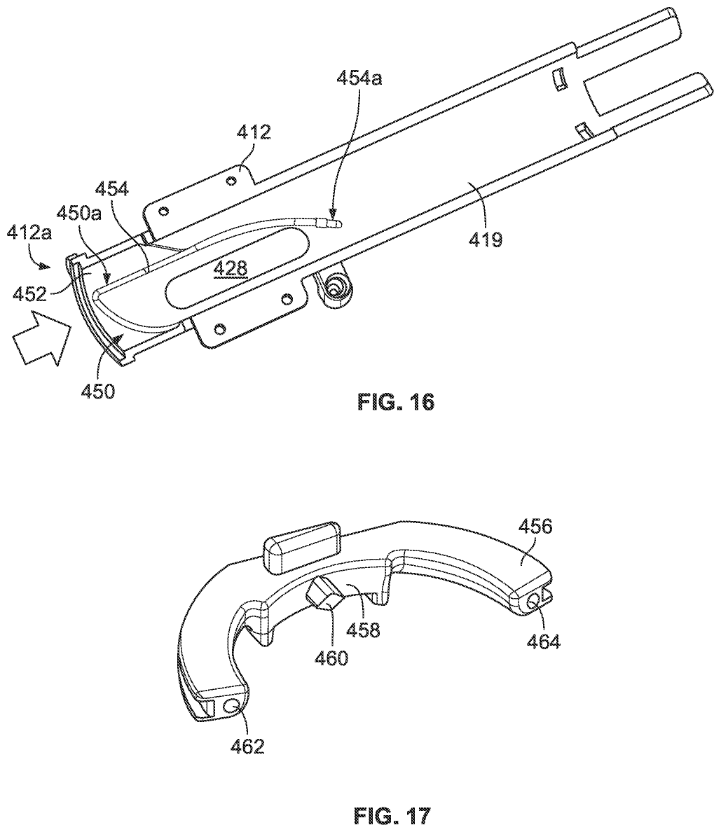

Turning to FIGS. 13a-23b, a device 400 having an alternative accessory grip 410 design is provided that includes similar features as the accessory grips 110, 210, 310 described in FIGS. 2a-12, and thus will not be described in substantial detail. Like the accessory grips 110 and 310, the accessory grip 410 defines a grip shell 412 having proximal and distal ends 412a, 412b, and a body 412c extending therebetween. The accessory grip 410 further includes a viewing window 428 positioned along the body 412c of the grip shell 412 and a lighting system 432 positioned at the proximal end 412a of the grip shell. The accessory grip 410 further includes a receiver (not shown), an informational display 434, an indirect start and feedback button 436, a skin sensor or sensors 438, and a sound feedback mechanism 440. Advantageously, and as illustrated in FIG. 14, the accessory grip 410 is designed such that the viewing window 428 allows approximately 70% of the dosage window 34 of the injector 10 to be viewable by a user.

With reference to FIGS. 15-18c, the device 400 is assembled by inserting the distal end 12b of the injector 10 upwardly into the accessory grip 410 from the proximal end 412a of the grip shell 412. As illustrated in FIG. 15, the throughbore 419 of the grip shell 412 includes any number of guide portions 450 in the form of a curved groove formed into the surface of the throughbore 419. The guide portion 450 includes a wide lower region 452 that tapers to a channel 454 having an end region 454a. Due to the bottom insertion of the injector 10, the channel 454 is positioned to pass the viewing window 428 until the end region 454a positioned generally above the viewing window 428.

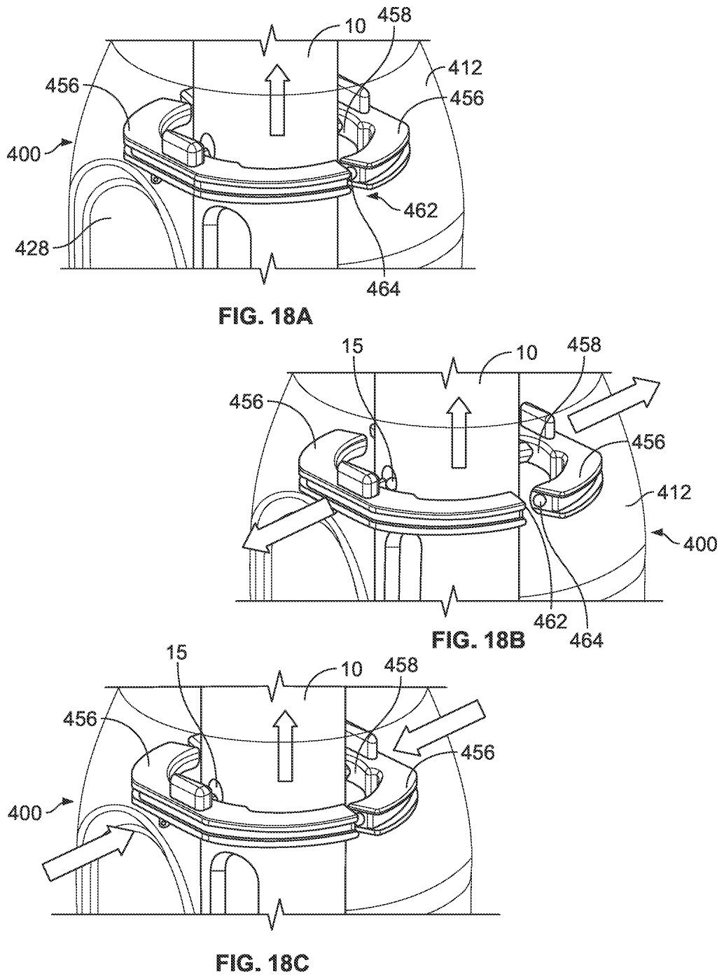

As shown in FIGS. 17a-18c, positioned at or near the end region 454a is any number (e.g., two) of locking members 456. As previously noted, the shell 12 of the injector housing 11 includes any number of bumps or protrusions 15 extending outwardly therefrom that engage the guide portion 450 during installation. The throughbore 419 of the grip shell 412 is dimensioned such that the protrusion or protrusions 15 may only be inserted into the throughbore 419 when they are positioned within the guide portion 450. By providing a relatively wide lower region 452, the injector 10 can be inserted into the accessory grip 410 in a misaligned configuration because continued insertion of the injector 10 into the throughbore 419 will cause the protrusion 15 to engage a sidewall 450a of the guide portion 450 and subsequently follow the shape of the tapered lower region 452 until the protrusion 15 is inserted into the channel 454, which results in proper orientation and alignment of the injector 10.

As previously noted, the locking member (or members) 456 is positioned at or near the end region 454a of the channel 454. The locking member 456 may be retained by and/or secured to the accessory grip 410 by any number of suitable approaches. The locking member 456 is in the form of a flexible and/or resilient ring that includes an inner surface 458 having a protrusion 460 that extends inwardly into the opening formed by the inner surface 458. As illustrated in FIG. 18a, during upwards insertion of the injector 10, the protrusion 15 of the injector 10 contacts the protrusion 460 of the locking member 456. As shown in FIG. 18b, continued upward insertion of the injector 10 causes the protrusion 15 of the injector 10 to urge the protrusion 460 of the locking member 456 outwardly until, as shown in FIG. 18c, the protrusion 15 of the injector 10 is positioned above the protrusion 460 of the locking member 456. Once the protrusion 15 of the injector passes the protrusion 460 of the locking member 456, the resilience of the locking member 456 causes the locking member 456 to move or snap to its initial position that restricts the injector 10 from being removed from the accessory grip. In the illustrated example, the locking members 456 further include a coupling mechanism in the form of a bump 462 and corresponding cavity 464 that engage each other when the locking members 456 are disposed in their initial position. Such an engagement further opposes axial forces and reduces the occurrence of the injector 10 decoupling from the accessory grip 410.

When the injector 10 is fully inserted into the accessory grip 410, the actuator button 32 abuts the indirect start and feedback button 436. The indirect start and feedback button 436 includes electronics such as a lighting or other feedback system that illuminates to alert a user when the device may be used. In one example, the indirect start and feedback button 436 may be in communication with the skin sensor 438 to provide a visual indication that the device 400 is properly positioned against the users skin. Upon pressing the indirect start and feedback button 436, the indirect start and feedback button 436 causes the actuator button 32 to be engaged to begin drug delivery.

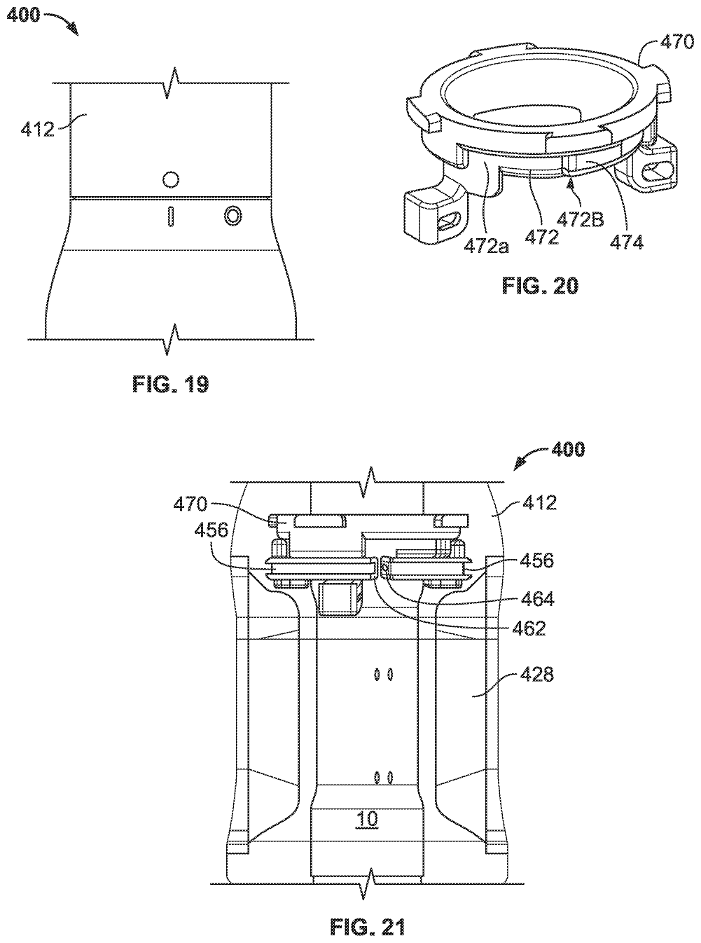

Turning to FIGS. 19-23b, upon administering the drug to the patient, the injector 10 can be readily removed by rotating the distal end 412b of the grip shell 412 approximately 30° relative to the proximal end 412a of the grip shell 412. As illustrated in FIG. 19, the body 412c of the grip shell 412 provides a visual indication for the relative positioning of the proximal and distal ends 412a, 412b to assist the user in discerning whether the injector 10 can be removed from the accessory grip 410. As illustrated in FIGS. 20-22b, the accessory grip 410 further includes a release mechanism 470 in the form of a ring that, upon rotating the proximal and distal ends 412a, 412b of the grip shell 412 relative to each other, causes the locking mechanisms 456 to separate until they no longer engage the protrusion 15 of the injector 10.

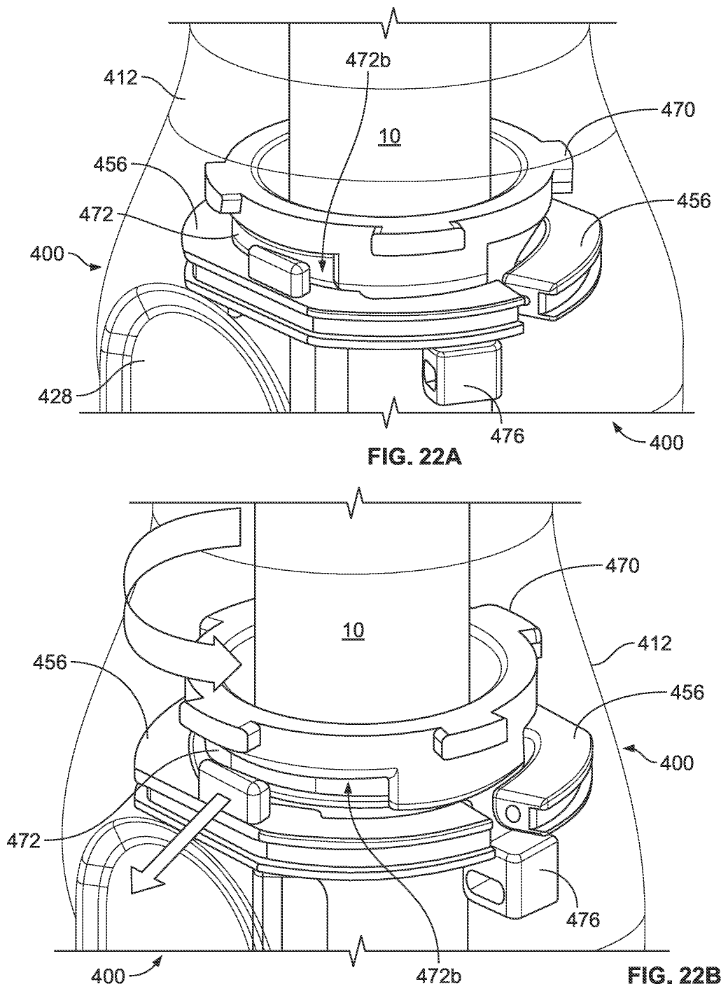

More specifically, the release ring 470 includes a contact surface 472 having a first portion 472a and a second portion 472b that accommodates an outwardly-protruding cam 474 and further includes a support ledge 476. As illustrated in FIGS. 21-22b, the locking mechanism 456 is disposed on the ledge 476, and the inner surface 458 of the locking mechanism 456 is positioned adjacent to the contact surface 472 of the release ring 470. As illustrated in FIG. 22a, in the initial configuration and during drug administration, the inner surface 458 of the locking mechanism 456 is positioned against the first portion 472a of the contact surface 472. Upon rotating the proximal end 412a of the grip shell 412 relative to the distal end 412b of the grip shell 412, the release ring 470 rotates relative to the locking mechanism 456 until the inner surface 458 of the locking mechanism 456 moves to the second portion 472b of the contact surface 472 and engages the outwardly protruding cam 474 positioned thereon. As shown in FIG. 22b, this engagement between the locking mechanisms 456 and the release ring 470 causes the locking mechanisms 456 to move outwardly until the protrusions 460 are no longer in engagement with the protrusions 15 disposed on the shell 12 of the injector housing 11.

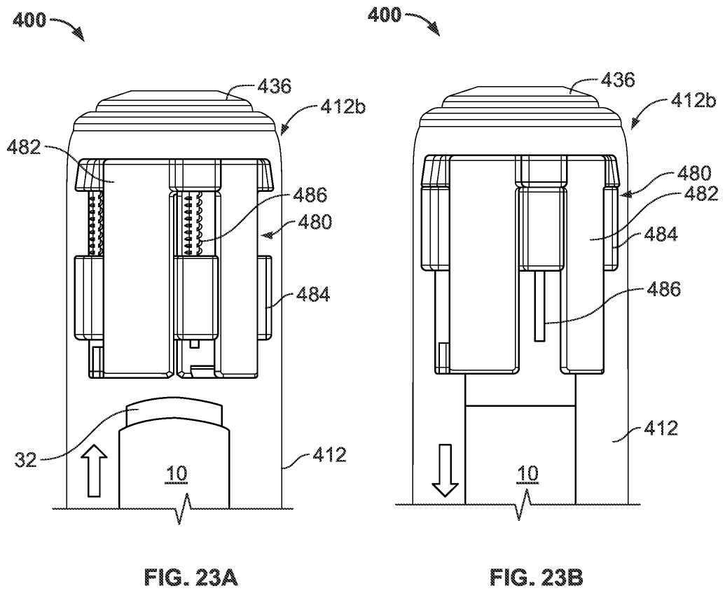

Turning to FIGS. 23a and 23b, an urging mechanism 480 is positioned at the distal end 412b of the grip shell 412 to gently urge the injector 10 out of the proximal end 412a of the grip shell 412. The urging mechanism 480 includes a cage 482, a moving platform 484, and an urging member 486. Upon inserting the injector 10 into the accessory grip 412, the distal end 12b of the shell 12 and/or the actuator button 32 moves the moving platform 484 upwards to a loaded position whereby the injector 10 is partially disposed within the cage 482. Upon actuation of the injector 10, the urging member 486 exerts a downward force on the moving platform 484 to push the injector downwards. In some approaches, the urging member 486 is in the form of a powered drive assembly being threadably engaged with the moving platform 484. In these examples, the urging force causes the urging member to rotate, thereby causing the moving platform 484 to move in an axial direction.

In other examples, the urging member 486 is in the form of a compression spring that is selectively coupled to the moving platform 484. When the injector 10 is inserted into the grip shell 412, the moving platform 484 may engage a catch that retains the moving platform 484 in the upper configuration depicted by FIG. 23b. Upon actuation of the device 400, the catch may disengage from the moving platform 484, thus allowing the urging member 486 to exert a downward force on the moving platform 484 that causes it to in turn push the injector 10. Other examples of urging members are possible.

The urging member 486 may exert the urging force either before or after the proximal and distal ends 412a, 412b of the grip shell 412 are rotated relative to each other in the previously-described manner. For example, while the moving platform 484 may begin exerting the force used to push the injector 10 out of the grip shell 412, this force may not be sufficient to overcome the force exerted on the injector 10 by the locking mechanism. As such, upon rotating the proximal and distal ends 412a, 412b of the grip shell 412, the moving platform 484 will then drive the injector 10 out of the grip shell 412. In other examples, actuating the urging member 486 may require a separate step that occurs after the user rotates the proximal and distal ends 412a, 412b of the grip shell 412.

So configured, the herein-described grip shells may be reusable by a patient, and accordingly, the described complex feedback systems may be incorporated into a patient's regimen while using single-use injectors 10. The grip shell 112 may be rechargeable to allow for continued usage. Grip shells 112, 212, 312, 412 of varying sizes may be provided to a healthcare provider to accommodate different user demographics. Additionally, grip shells 112 having varying desired smart functionality may be provided for patients with different technological expertise. The devices 100, 200, 300, 400 may be configured for users having different characteristics (e.g., age, skill set, experience level, etc.) to facilitate ease of use, and thus greater adherence to treatment guidelines by decreasing a risk of premature lifting through comfortable handling and clear injection status feedback. This in turn may improve therapy outcomes. The connectivity features described herein may further improve use compliance and ease of use of the devices.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACl-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure.

In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
an injector including:
an injector housing defining a housing body having a proximal end, a distal end, and a longitudinal axis extending between the proximal end and the distal end thereof;
a needle assembly at least partially disposed within the injector housing at the proximal end thereof, the needle assembly comprising a syringe barrel containing a medicament and a needle or a cannula;
a drive assembly at least partially disposed within the housing and operably coupled to the needle assembly to urge the medicament through the needle or cannula; and
an accessory grip defining a grip shell having a proximal end, a distal end, and a body extending between the proximal end and the distal end thereof, the proximal end of the grip shell including a first opening dimensioned to receive a first portion of the injector housing, the distal end of the grip shell including a second opening dimensioned to receive a second portion of the injector housing.

2. The drug delivery device of claim 1, further comprising:
an injector housing latching member disposed on the housing body; and
a grip latching member disposed on the grip shell, wherein the grip latching member secures to the injector housing latching member to secure the injector housing to the accessory grip.

3. The drug delivery device of claim 2, wherein the injector housing latching member comprises at least one depression formed on the shell, and wherein the grip latching member comprises at least one finger member configured to be inserted into the at least one depression.

4. The drug delivery device of claim 3, wherein the at least one depression forms a dosage window on the injector housing.

5. The drug delivery device of claim 4, wherein the accessory grip further includes at least one viewing window that is aligned with the dosage window.

6. The drug delivery device of claim 1, wherein the grip shell further defines a throughbore extending between the first opening and the second opening thereof, the throughbore being dimensioned to receive the injector such that the proximal end of the injector housing is exposed through the first opening of the grip shell and the distal end of the injector housing is exposed through the second opening of the grip shell.

7. The drug delivery device of claim 1, wherein the accessory grip further comprises at least one electronic device at least partially disposed within the grip shell.

8. The drug delivery device of claim 7, wherein the at least one electronic device comprises at least one of:
a display;
a lighting system;
a skin sensor;
a communications module;
a motion sensor; or
an electromechanical feedback mechanism.

9. The drug delivery device of claim 8, wherein the at least one electronic device is adapted to communicate with the injector.

10. The drug delivery device of claim 1, wherein the accessory grip comprises a tubular clamshell that extends in a direction along the longitudinal axis of the injector housing.

11. The drug delivery device of claim 1, wherein the accessory grip comprises an elongated dome-shaped clamshell having a curved upper gripping surface.

12. The drug delivery device of claim 1, wherein the proximal end of the grip portion further comprises a planar contact surface.

13. An accessory grip for a drug delivery device, the accessory grip comprising:
a grip shell having a proximal end, a distal end, and a body extending between the proximal end and the distal end;
a first opening formed at the proximal end of the grip shell, the first opening dimensioned to receive a first portion of a drug delivery device;
a second opening formed at the distal end of the grip shell, the second opening dimensioned to receive a second portion of the drug delivery device, the first opening and the second opening being in communication with each other via a throughbore extending therebetween; and
a grip latching member disposed on the grip shell, wherein the grip latching member is adapted to couple to the drug delivery device to secure the accessory grip to the drug delivery device.

14. The accessory grip of claim 13, further comprising at least one viewing window disposed on the body of the grip shell.

15. The accessory grip of claim 13, further comprising at least one electronic device at least partially disposed within the grip shell.

16. The accessory grip of claim 15, wherein the at least one electronic device comprises at least one of:
a display;
a lighting system;
a skin sensor;
a communications module;
a motion sensor;
an electromechanical feedback mechanism.

17. The accessory grip of claim 13, wherein the grip shell comprises a tubular clamshell that extends in a direction along a longitudinal axis.

18. The accessory grip of claim 13, wherein the grip shell comprises an elongated dome-shaped clamshell having a curved upper gripping surface.

19. The accessory grip of claim 13, wherein the proximal end of the grip portion further comprises a planar contact surface.
